(19) United States
(12) Patent Application Publication
(10) Pub. No.: US 2024/0066191 A1
(43) Pub. Date: Feb. 29, 2024

(54) Breast Milk Pump Bottle Securing Device

(71) Applicants: Danielle Grueninger, Mt. Vernon, IN (US); Billy Butler, Mt. Vernon, IN (US)

(72) Inventors: Danielle Grueninger, Mt. Vernon, IN (US); Billy Butler, Mt. Vernon, IN (US)

(21) Appl. No.: 17/895,185

(22) Filed: Aug. 25, 2022

(51) Int. Cl. A61M 1/06

(52) U.S. Cl. CPC A61M 1/062

(57) ABSTRACT

A breast milk pump bottle securing device for encasing a bottle and securing to a breast milk pump includes a sleeve enwrapping a bottle for breast milk. The sleeve has an exterior surface and an interior surface. Furthermore, the sleeve has an open end and a base end. A base is positioned on the base end, thus the base and the interior surface of the sleeve enclose an interior. A strap is positioned on a perimeter of the open end of the sleeve and extends out from the open end. The strap has a button end, and the button end has a button. The strap is folds over a top of a breast milk pump. A clasp is positioned on the exterior surface of the sleeve. The clasp is engages by snap button fastening to the button.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to breast milk pump securing device and more particularly pertains to a new breast milk pump securing device for encasing a bottle and securing to a breast milk pump.

### (2) Description of Related Art Including Information Disclosed Under 37 CFR 1.97 and 1.98

The prior art relates to breast milk pump securing devices. The prior art relates to a variety of breast milk pump securing devices configured for assisting in securing the bottle to the breast milk pump. Known prior art lacks a breast milk pump securing device being removable and being configured for enwrapping the bottle prior to securing to the breast milk pump.

### BRIEF SUMMARY OF THE INVENTION

An embodiment of the disclosure meets the needs presented above by generally comprising a sleeve configured for enwrapping a bottle for breast milk. The sleeve has an exterior surface and an interior surface. Furthermore, the sleeve has an open end and a base end. A base is positioned on the base end, wherein the base and the interior surface of the sleeve enclose an interior. A strap is positioned on a perimeter of the open end of the sleeve and protrudes out from the open end. The strap has a button end, and the button end has a button. The strap is configured for folding over a top of a breast milk pump. A clasp is positioned on the exterior surface of the sleeve. The clasp is configured for engaging by snap button fastening to the button.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front isometric view of a breast milk pump bottle securing device according to an embodiment of the disclosure.

FIG. 2 is a front view of an embodiment of the disclosure.

FIG. 3 is a side view of an embodiment of the disclosure.

FIG. 4 is an in-use view of an embodiment of the disclosure.

FIG. 5 is an in-use view of an embodiment of the disclosure.

FIG. 6 is an in-use view of an embodiment of the disclosure.

FIG. 7 is a front isometric view of an alternate embodiment of the disclosure.

FIG. 8 is an in-use view of an alternate embodiment of the disclosure.

### DETAILED DESCRIPTION OF THE INVENTION

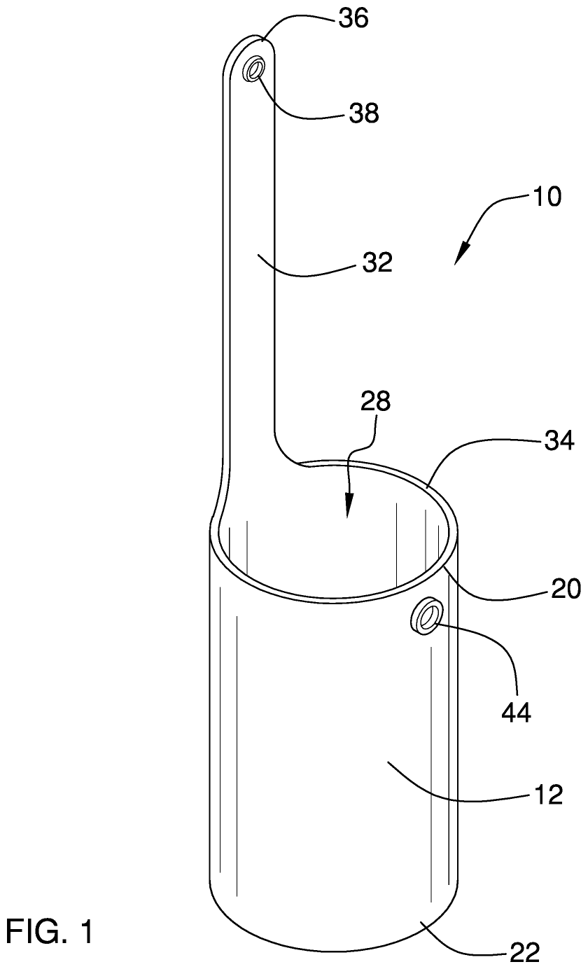

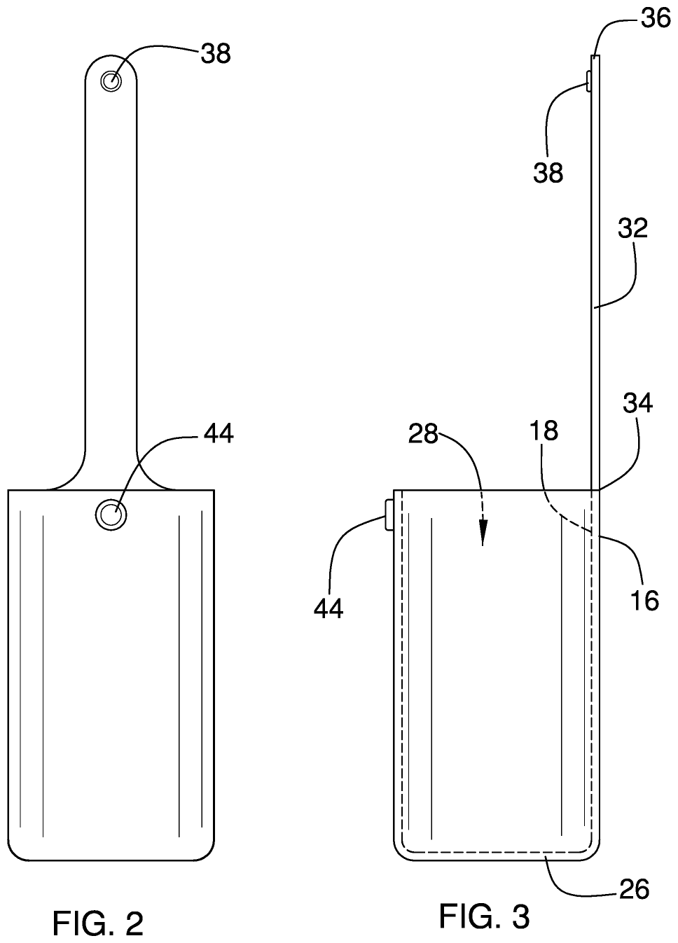

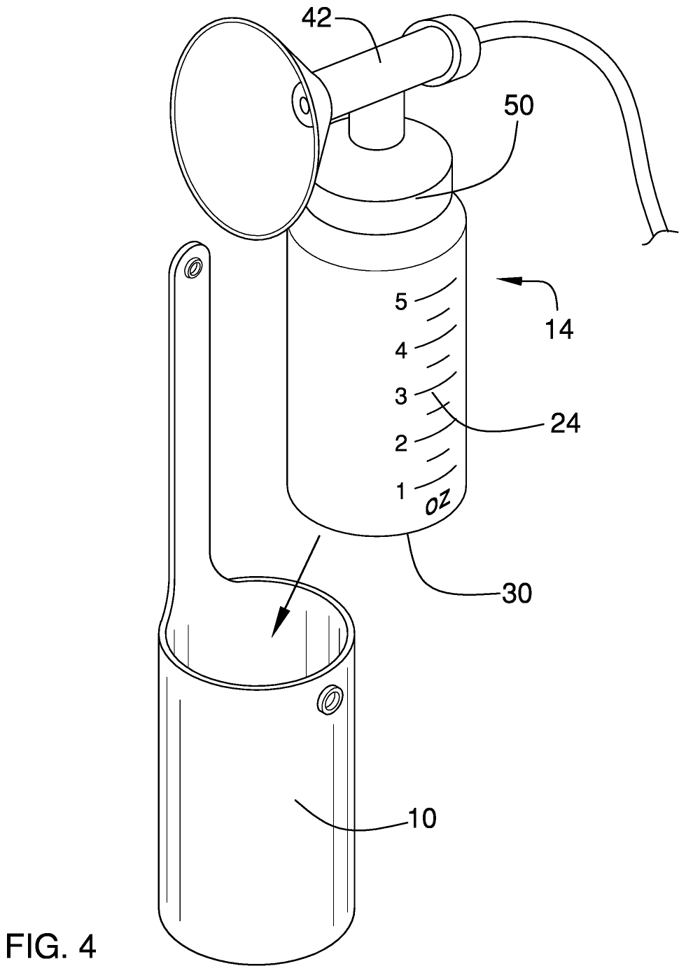

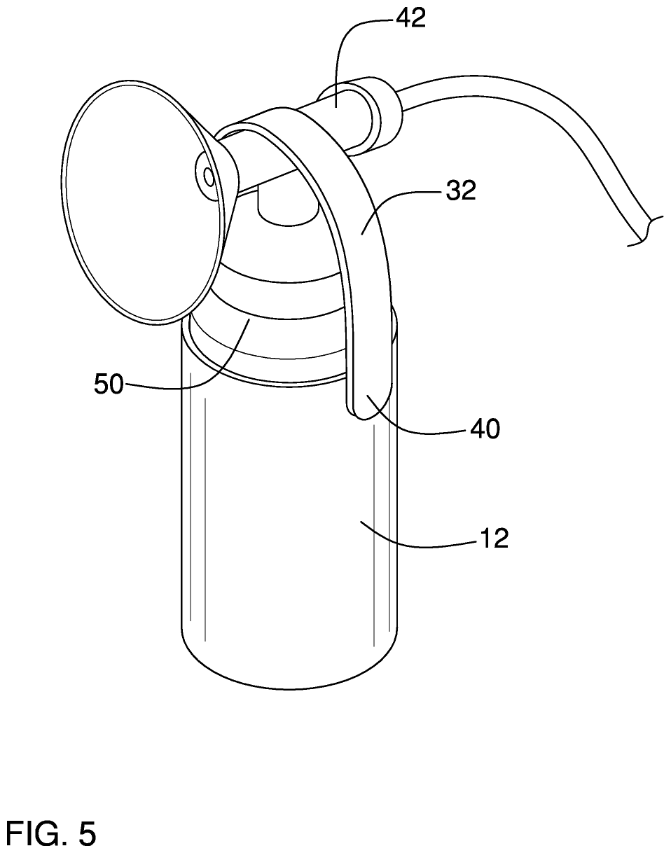

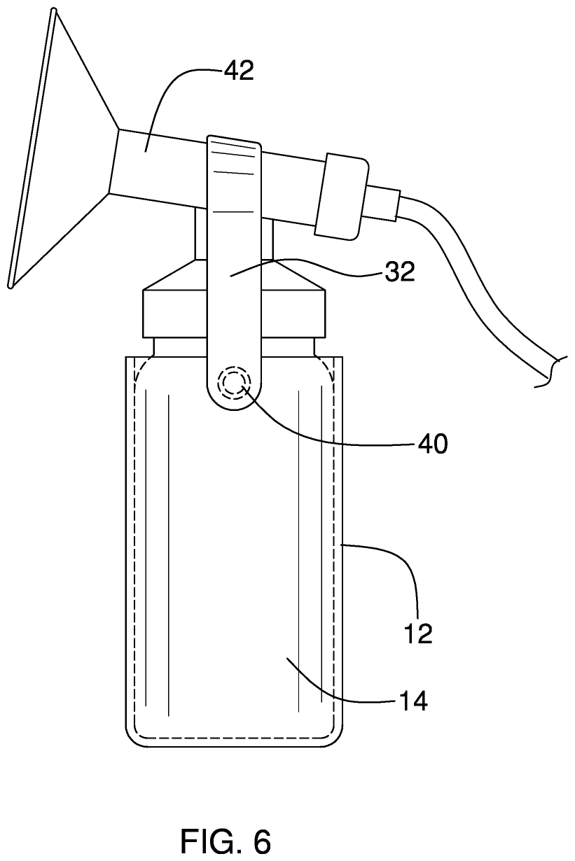

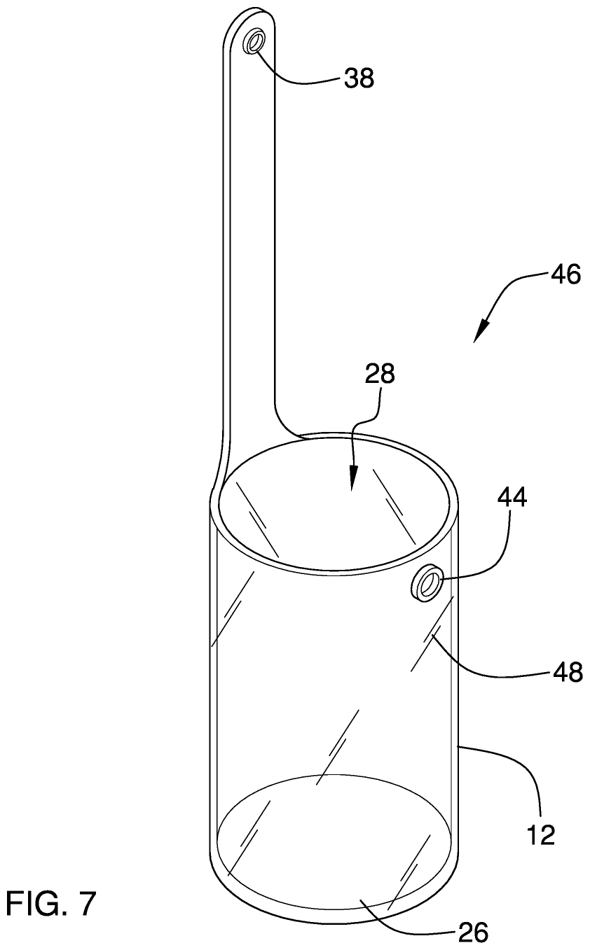

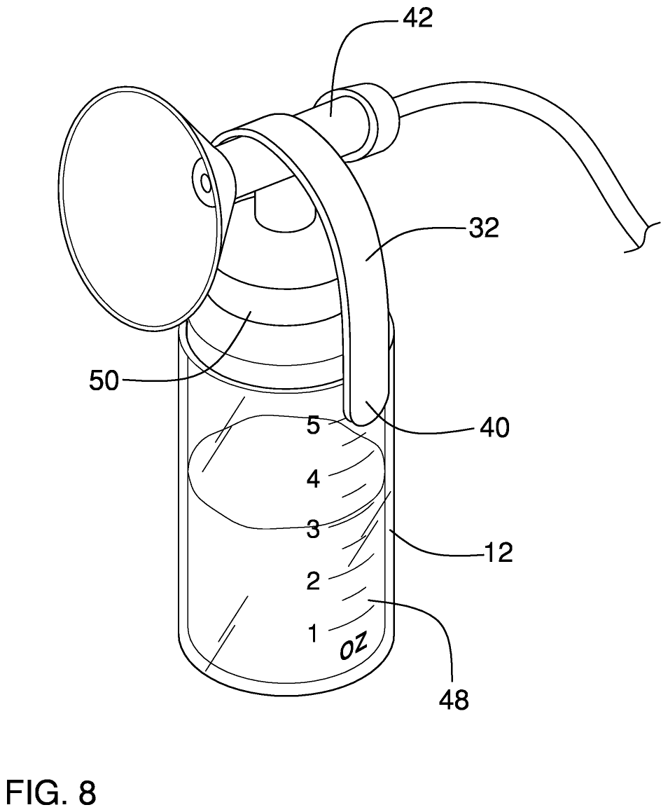

With reference now to the drawings, and in particular to FIGS. 1 through 8 thereof, a new breast milk pump securing device embodying the principles and concepts of an embodiment of the disclosure and generally designated by the reference numeral 10 will be described.

As best illustrated in FIGS. 1 through 8, the breast milk pump bottle securing device 10 generally comprises a sleeve 12. The sleeve 12 is configured for enwrapping a bottle 14 for breast milk. The sleeve 12 is removable from the bottle 14. The sleeve 12 has a tubular shape, wherein the sleeve 12 has an exterior surface 16 and an interior 28 surface 18. The sleeve 12 has an open end 20 and a base end 22. The open end 20 and the base end 22 are positioned parallel relative to each other. The sleeve 12 is configured for concealing the body 24 of the bottle 14.

A base 26 is positioned on the base end 22. The base 26 is circular and is complementary to the tubular shape of the sleeve 12. The base 26 and the interior 28 surface 18 of the sleeve 12 enclose an interior 28. The interior 28 defines a space for the bottle 14 to be nested within. The base 26 is configured for being positioned abutting a bottom 30 of the bottle 14, wherein the top 50 of the bottle 14 is positioned in the open end 20 of the sleeve 12. A strap 32 is positioned on a perimeter 34 of the open end 20 of the sleeve 12 and protrudes out from the open end 20. The strap 32 has a button end 36, and the button end 36 has a button 38. The button 38 is a first portion of a snap button fastener 40. The strap 32 is configured for folding over a top of a breast milk pump 42 and positioning the button end 36 of the strap 32 proximate to the exterior of the sleeve 12.

A clasp 44 is positioned on the exterior surface 16 of the sleeve 12. The clasp 44 is positioned proximate to the perimeter 34 of the open end 20 of the sleeve 12. Furthermore, the clasp 44 is positioned parallel relative to the strap 32 on the perimeter 34 of the open end 20. The clasp 44 is a second portion of the snap button fastener 40, wherein the clasp 44 is complementary to the button 38. The clasp 44 is configured for engaging by snap button fastening to the button 38.

An alternate embodiment 46 of the breast milk pump bottle securing device 10 includes the sleeve 12 of the alternate embodiment 46 being a transparent material 48. The transparent material 48 is configured for allowing a user to view the interior 28 of the sleeve 12 from the exterior surface 16. The alternate embodiment 46 allows the user to view the bottle 14 within the sleeve 12, wherein the user can determine the capacity level of breast milk within the bottle 14 while engaging with the breast milk pump 42.

In use, the user nests the body 24 of the bottle 14 for breast milk in the interior 28 of the sleeve 12, with the top of the bottle 14 exposed at the open end 20 of the sleeve 12. The user attaches the top 50 of the bottle 14 to the breast milk pump 42. Subsequently, the user folds over the strap 32 from the perimeter 34 of the open end 20 and secure the button end 36 of the strap 32 to a clasp 44 of the sleeve 12 by snap button fastening. The bottle 14 remains secure within the sleeve 12 while in use.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A breast milk pump bottle securing device configured for securing a bottle to a breast milk pump, the breast milk pump bottle securing device comprising:
a sleeve being configured for enwrapping a bottle for breast milk, said sleeve having an exterior surface and an interior surface, said sleeve having an open end and a base end;
a base being positioned on said base end, said base and said interior surface of said sleeve enclosing an interior;
a strap being positioned on a perimeter of said open end of said sleeve and protruding out from said open end, said strap having a button end, said button end having a button, said strap being configured for folding over a top of a breast milk pump; and
a clasp being positioned on said exterior surface of said sleeve, said clasp being configured for engaging by snap button fastening to said button.

2. The breast milk pump bottle securing device of claim 1, further comprising said sleeve being removable from the bottle.

3. The breast milk pump bottle securing device of claim 2, further comprising said sleeve having a tubular shape.

4. The breast milk pump bottle securing device of claim 3, further comprising said open end and said base end being positioned parallel relative to each other.

5. The breast milk pump bottle securing device of claim 4, further comprising said sleeve being configured for concealing the body of the bottle.

6. The breast milk pump bottle securing device of claim 5, further comprising said base being circular.

7. The breast milk pump bottle securing device of claim 6, further comprising said interior defining a space for the bottle to be nested within, said base being configured for being positioned abutting a bottom of the bottle wherein a top of the bottle being positioned in said open end of said sleeve.

8. The breast milk pump bottle securing device of claim 7, further comprising said button being a first portion of a snap button fastener.

9. The breast milk pump bottle securing device of claim 8, further comprising said clasp being positioned proximate to said perimeter of said open end of said sleeve, said clasp being positioned parallel relative to said strap on said perimeter of said open end.

10. The breast milk pump bottle securing device of claim 9, further comprising said clasp being a second portion of said snap button fastener, said clasp being complementary to said button.

11. The breast milk pump bottle securing device of claim 1, further comprising an alternate embodiment of said breast milk pump bottle securing device, said sleeve of said alternate embodiment being a transparent material, said transparent material being configured for allowing a user to view said interior of said sleeve from said exterior surface.

12. A breast milk pump bottle securing device configured for securing a bottle to a breast milk pump, the breast milk pump bottle securing device comprising:
a sleeve being configured for enwrapping a bottle for breast milk, said sleeve being removable from the bottle, said sleeve having a tubular shape, said sleeve having an exterior surface and an interior surface, said sleeve having an open end and a base end, said open end and said base end being positioned parallel relative to each other, said sleeve being configured for concealing the body of the bottle;
a base being positioned on said base end, said base being circular, said base and said interior surface of said sleeve enclosing an interior, said interior defining a space for the bottle to be nested within, said base being configured for being positioned abutting a bottom of the bottle wherein a top of the bottle being positioned in said open end of said sleeve;
a strap being positioned on a perimeter of said open end of said sleeve and protruding out from said open end, said strap having a button end, said button end having a button, said button being a first portion of a snap button fastener, said strap being configured for folding over a top of a breast milk pump;
a clasp being positioned on said exterior surface of said sleeve, said clasp being positioned proximate to said perimeter of said open end of said sleeve, said clasp being positioned parallel relative to said strap on said perimeter of said open end, said clasp being a second portion of said snap button fastener, said clasp being complementary to said button, said clasp being configured for engaging by snap button fastening to said button; and
an alternate embodiment of said breast milk pump bottle securing device, said sleeve of said alternate embodiment being a transparent material, said transparent material being configured for allowing a user to view said interior of said sleeve from said exterior surface.
